(19) United States
(12) Patent Application Publication
(10) Pub. No.: US 2005/0004446 A1
(43) Pub. Date: Jan. 6, 2005

(54) Model assisted planning of medical imaging

(76) Inventors: Brett Cowan, Auckland (NZ); Thomas O'Donnell, New York, NY (US); Alistair Young, Auckland (NZ)

Correspondence Address: Siemens Corporation;Intellectual Property Department, 170 Wood Avenue South, Iselin, NJ, 08830, US

(21) Appl. No.: 10/876,211

(22) Filed: Jun. 24, 2004

Related U.S. Application Data
(60) Provisional application No. 60/482,328, filed on Jun. 25, 2003.

(51) Int. Cl. A61B 5/05

(52) U.S. Cl. 600/407; 128/920

(57) ABSTRACT

A method and system for medical image acquisition are provided, where the method includes acquiring an image of the region of interest, acquiring a model for a region of interest, and fitting the model to the image; the system includes a modeling unit for modeling a region of interest; an acquisition unit in signal communication with the modeling unit for acquiring an image of the region of interest; and a fitting unit in signal communication with the acquisition unit for fitting the model to the image.

### CROSS-REFERENCE TO RELATED APPLICATION

This application claims the benefit of U.S. Provisional Application Ser. No. 60/482,328 (Attorney Docket No. 2003P09206US), filed on 25 Jun. 2003 and entitled “Model Assisted Planning of Medical Imaging”, which is incorporated herein by reference in its entirety..

### BACKGROUND OF INVENTION

1. Technical Field

The present invention relates to medical imaging, and more particularly, to determining a plan for acquiring medical images of a desired region.

2. Discussion of the Related Art

Certain body regions require scan planning in order to acquire views that illuminate an area of interest. The left ventricle of the heart, for example, is often studied from the short-axis view. Given a set of axial scout images, traditional planning for a short axis series is a time-consuming two-step process, not easily performed by beginners. First, a single long-axis oblique scout is acquired. From that, a second (double) oblique scout is taken. The short axis series is then planned on the double oblique scout.

The same is true when imaging other areas of the body. When imaging the brain, for example, physicians often wish to orient the scan parallel to the base of the skull. In the kidneys, images aligned with the natural long and short axes of the organ are desirable. A means of automating the complex pre-scanning phase and thereby increase the reproducibility and reliability of acquisition planning is desirable.

### SUMMARY OF THE INVENTION

An exemplary embodiment of the present invention includes a method of medical image acquisition. The method comprises acquiring an image and a model for a region of interest. This model is fit to the image.

Another exemplary embodiment of the present invention includes an apparatus for medical image acquisition. The apparatus comprises an acquisition means for acquiring an image of the region of interest. It comprises a modeling means, in signal communication with the acquisition means, for modeling a region of interest. It also comprises a fitting means, in signal communication with the acquisition means, for fitting the model to the image.

Another exemplary embodiment of the present invention includes a system for medical image acquisition. The system comprises a modeling unit for modeling a region of interest. There is also an acquisition unit, in signal communication with the modeling unit, for acquiring an image of the region of interest. There is also a fitting unit, in signal communication with the acquisition unit, for fitting the model to the image.

Another exemplary embodiment of the present invention includes a program storage device readable by machine, tangibly embodying a program of instructions executable by the machine to perform a method of medical image acquisition. The program steps comprise acquiring an image and a model for a region of interest. This model is fit to the image.

### BRIEF DESCRIPTION OF THE DRAWINGS

FIG. 1 is a schematic diagram showing an exemplary embodiment of a computer system;

FIG. 2A is a medical image depicting an exemplary embodiment of the current invention where a 3D model wire frame of the Left Ventricle (“LV”) of a human heart is fitted to two MR scout images taken from different orientations;

FIG. 2B is a medical image depicting an exemplary embodiment of the current invention where a 3D model wire frame of the LV of a human heart is depicted;

FIG. 3 is a medical image depicting an exemplary embodiment of the current invention where a MR image of a heart and the planned locations for future scans based on the 3D LV model fitted to the image are shown;

FIG. 4 is a medical image depicting an exemplary embodiment of the current invention where a model is being fitted to a 2D MR image;

FIG. 5 is a medical image depicting an exemplary embodiment of the current invention where an MR image has been filtered using a Sobel filter;

FIG. 6 is a flow diagram depicting an exemplary embodiment of the current invention; and

FIG. 7 is schematic diagram of an exemplary embodiment of a system implementing the current invention.

### DETAILED DESCRIPTION OF PREFERRED EMBODIMENTS

Exemplary embodiments of the present invention provide methods, systems, and apparatus for streamlining scan planning for regions of interest. The images used can be acquired using a Magnetic Resonance Scanner (“MR”), a Positron Emission Tomography Scanner (“PET”), a Single Photon Emission Computed Tomography (“SPECT”), a Computed Tomography Scanner (“CT”), and/or other medical imaging devices. CT, SPECT, and PET volume data of the region of interest, among other data sources representative of the region, can be reformatted, subsequent to acquisition, to create the desired images as well. After the viewing planes have been determined, the images can be rescanned or the data, like that of CT volumes, can be reformatted to acquire new images at the new viewing planes.

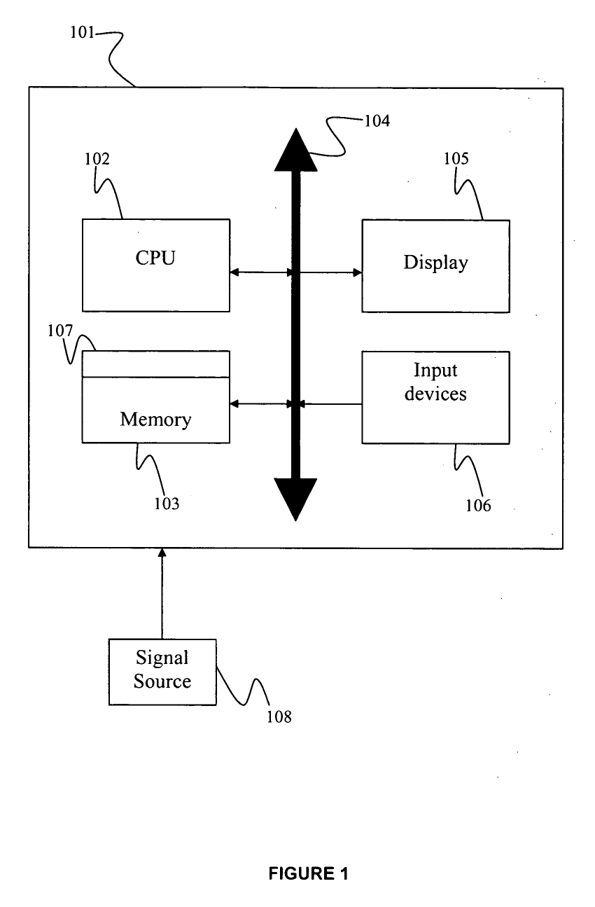

Referring to FIG. 1, according to an exemplary embodiment of the present invention, a computer system 101 for implementing the present invention includes a central processing unit (“CPU”) 102, a memory 103 and an input/output (“I/O”) interface 104. The computer system 101 is generally coupled through the I/O interface 104 to a display 105 and various input devices 106 such as a mouse, keyboard, and medical imaging devices. The support circuits can include circuits such as cache, power supplies, clock circuits, and a communications bus. The memory 103 can include random access memory (“RAM”), read only memory (“ROM”), disk drive, tape drive, etc., or a combination thereof. The present invention can be implemented as a routine 107 that is stored in memory 103 and executed by the CPU 102 to process the signal from the signal source 108. As such, the computer system 101 is a general-purpose computer system that becomes a specific purpose computer system when executing the routine 107 of the present invention.

The computer system 101 also includes an operating system and microinstruction code. The various processes and functions described herein may either be part of the microinstruction code or part of the application program (or a combination thereof), which is executed via the operating system. In addition, various other peripheral devices may be connected to the computer platform, such as an additional data storage device and a printing device.

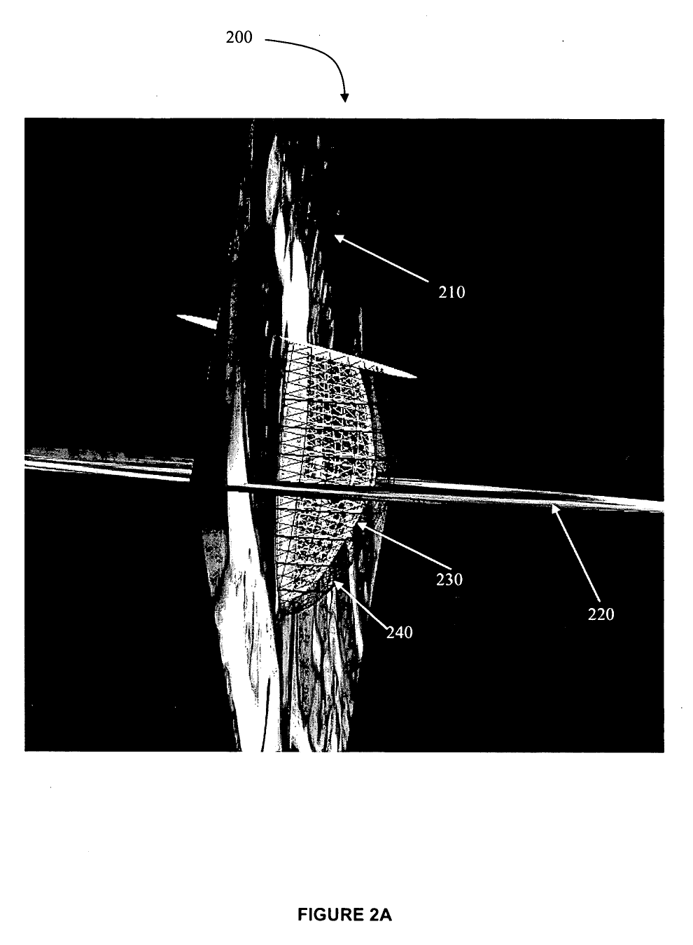

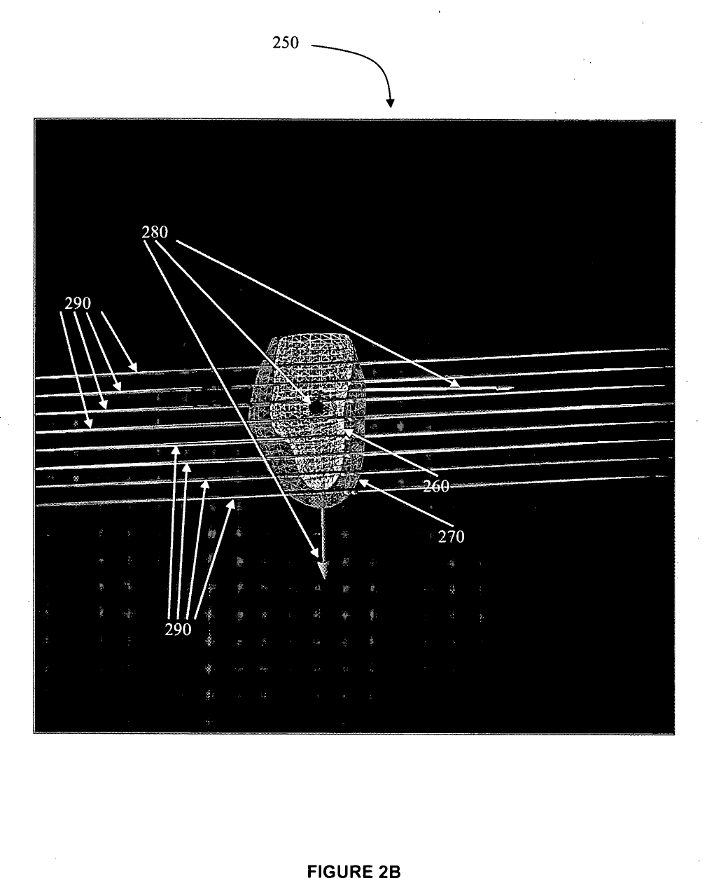

FIG. 2A is a medical image depicting an exemplary embodiment of the current invention, and is indicated generally by reference numeral 200. Here reference numerals 230 and 240 point out a 3D model wire frame of the Left Ventricle (“3D LV”) of a human heart. Reference numeral 230 represents a first portion of the 3D LV model and reference numeral 240 represents a second portion of the 3D LV model. Reference numerals 210 and 220 point out two MR scout images taken from different orientations to which the model 230 and 240 are fitted.

FIG. 2B is a medical image depicting an exemplary embodiment of the current invention, and is indicated generally by reference numeral 250. Here the 3D LV model depicted by reference numerals 230 and 240 is presented. Reference numeral 260 represents the first portion of the 3D LV model and reverence numeral 270 represents the second. Reference numeral 280 points to the three dimensional axis associated with the 3D LV model 260 and 280. Reference numeral 290 points to eight possible scan image locations that cut through the model.

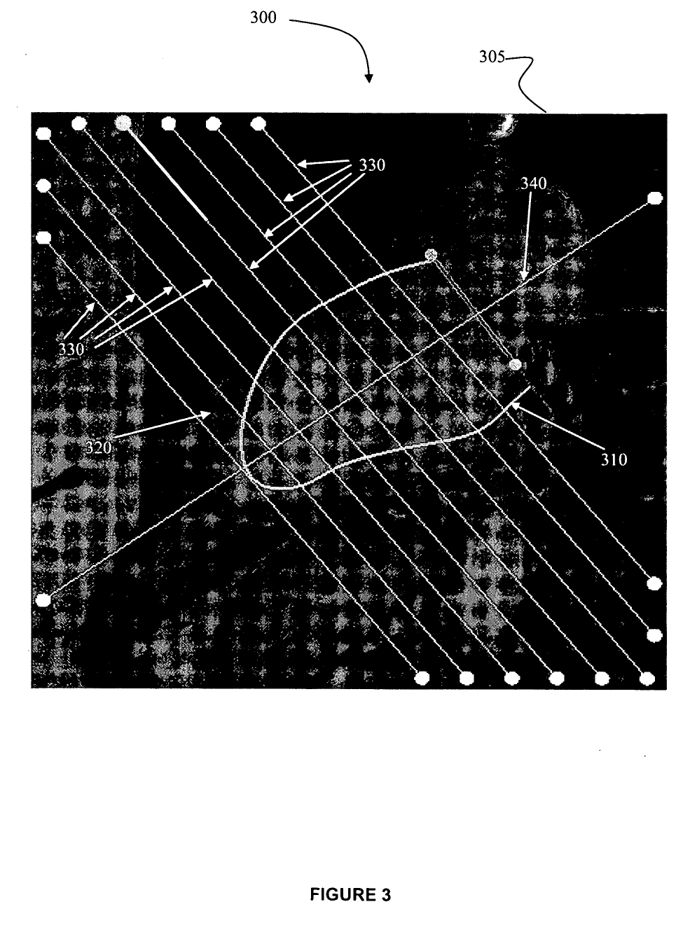

FIG. 3 is a medical image depicting an exemplary embodiment of the current invention, and is indicated generally by reference numeral 300. The 3D LV model depicted in FIG. 2A by reference numerals 230 and 240 (comprising of the first portion 310 and the second portion 320) is used to plan the locations of future scans. Reference numeral 330 represents a set of planned locations for future scans, where each line represents a different parallel slice of the heart that is to be imaged. Reference numeral 340 represents another planned location for a future scan of the heart. Reference numeral 305 is a MR image of the heart that the 3D LV model is modeling and fitted to.

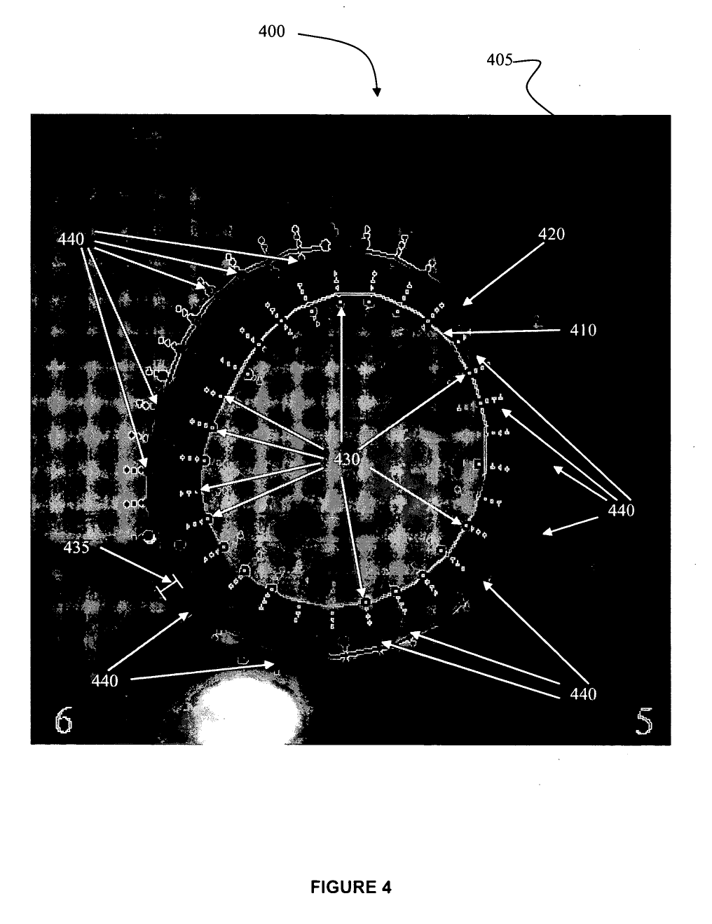

FIG. 4 is a medical image depicting an exemplary embodiment of the current invention, and is indicated generally by reference numeral 400. Here the 3D LV model discussed earlier is being fitted to the 2D MR image of a heart 405. Reference numeral 410 points to the first portion of the model and reference numeral 420 points to the second. Reference numeral 430 point to a representative set of the points used to delineate a first border of the LV of a heart to which the first portion of the model 410 is fitted. Reference numeral 440 point to a representative set of points used to delineate a second border of the LV of a heart to which the second portion of the model 420 is fitted. The distance between the selected points 440 and 430, representing the delineated border, and the model 410 and 420 is the Root Mean Square (“RMS”) distance. The line pointed to by reference numeral 435 illustrates an example of such a distance.

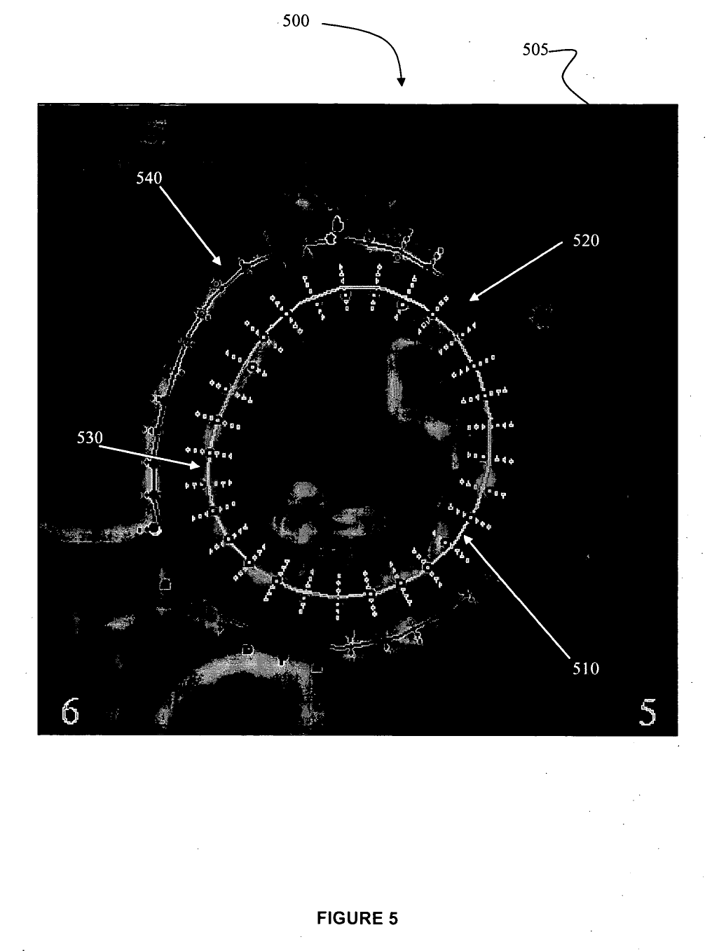

FIG. 5 is a medical image depicting an exemplary embodiment of the current invention, and is indicated generally by reference numeral 500. Here the MR image 505 depicts the results of filtering the MR image 405 through a Sobel edge detection filter. The filter helps highlight a first border of the LV 530 and a second border of the LV 540. Reference numeral 510 points to the first portion of the 3D LV model 230 depicted in FIG. 2A. Reference numeral 520 points to the second portion of the 3D LV model 240 depicted in FIG. 2A. The first portion of the model 510 is fitted to the first LV border 530 and the second portion of the model 520 is fitted to the second LV border 540.

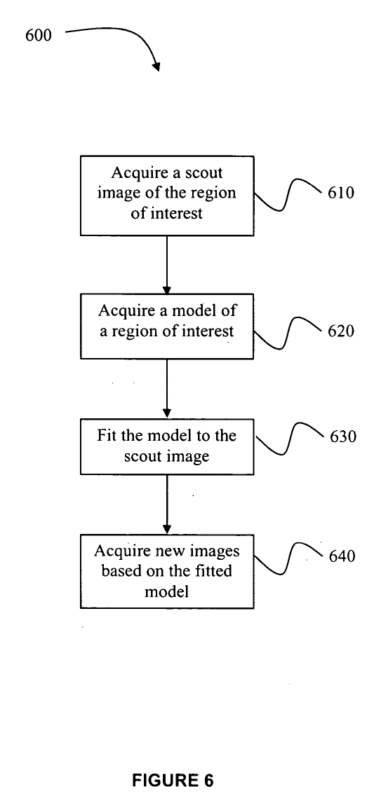

FIG. 6 is a flow diagram that depicts an exemplary embodiment of the current invention, and is indicated generally by reference numeral 600. Block 610 depicts the step of acquiring a scout image of the region of interest, which can be an axial image. A medical imaging device may take the scout image or existing data is reformatted to produce the scout image; an exemplary embodiment of such an image is depicted by reference numeral 405.

Block 620 represents the step of acquiring a model of the region of interest. These models depict different areas of interest, including the heart and lungs; an exemplary embodiment of such a model is the 3D LV model identified by reference numerals 260 and 270 in FIG. 2B. Each model, among other characteristics, also has an associated coordinate system that can be used to help acquire future images. An exemplary embodiment of such a coordinate system is identified by reference numeral 280 in FIG. 2B.

Block 630 depicts the step of fitting the model to the scout image. This can be done manually, semi-automatically, or automatically and need not be precise. For example, in the case of scanning the left ventricle, fixing the general pose, long and short axis orientations would be sufficient. Subsequent image acquisitions can be based on the coordinate system associated with the model. An exemplary embodiment of fitting a model to a scout image is depicted in FIG. 4.

Block 640 depicts the step of acquiring additional images of the area of interest. These new images can be taken from new medical scans or by reformatting existing data sets. These new images are based on a coordinate system associated with the model; an exemplary embodiment of such a coordinate system is identified by reference numeral 280 in FIG. 2B. For example, in the case of the scan of the left ventricle discussed earlier, since both the short axis orientation of the model and its extent (apex to base distance) is known analytically, optimally spaced short axis planes may be specified. The number of slices and their positions may be based on configurable defaults for slices, spacing, position, etc. It now becomes possible to specify a standard acquisition such as “left atrial series” or “aortic flow” and receive the standard radiological acquisitions in a reliable, efficient manner. For dynamic regions, such as the heart, it also becomes possible to temporally adjust the scan positions to follow a region over time.

An exemplary embodiment of such a step is depicted in FIG. 3 where reference numerals 330 and 340 identify a set of scan positions. These scan positions represent the positions of images to be acquired in relation to a model 260 and 270 fitted to a scout image 405.

To model and fit the structures of interest, many approaches may be employed. In an exemplary embodiment of the current invention, a clinician will delineate the borders of the region of interest in at least one 2D scout images using a contour drawing tool such as Argus™, by Siemens Medical Solutions; an example delineating the borders is illustrated by reference numerals 430 and 440 in FIG. 4. A 3D parametric model may then be fit to this set of 2D contours (an example of fitting a model to an identified contour is illustrated by reference numerals 410 and 420 in FIG. 4). The parametric model, in the simplest case, could be a 3D ellipsoid with parameters describing the radii in the model-centered x, y, and z directions (an example of model-centered x, y, and z directions is identified by reference numeral 280 in FIG. 2B). These parameters are adjusted to minimize the Root Mean Square (“RMS”) distance calculable between the delineated border, and therefore the contour points, and the surface of the model (an example of such a distance is identified by reference numeral 435 in FIG. 4). This minimization may employ gradient decent, if the parametric model is in analytic form. In any case, the range of parameter values is searched to find the settings, which place the model closest to the data.

In another exemplary embodiment of the current invention, the model may be of polygonal form and may be fit by treating the polygons as forming a spring-node mesh. More specifically, starting with a polygonal model, which resembles a typical instance of the structure of interest, the shape of the model is changed by adjusting the vertices, also known as nodes, of the polygons so as to minimize the RMS distance between the delineated contour points on the scout image and the surface of the model. In order to maintain a smooth model surface, the sides of the polygons act like springs so that, when one node or vertex is adjusted, its neighbors are pulled along.

In another exemplary embodiment of the current invention, in the case where a clinician is not available to manually delineate the borders in the scout image, edge detection algorithms may be employed. In one approach, the scout image is convolved with a filter, e.g., a Sobel filter, which detects sharp changes in intensity, indicating the borders of the region of interest. Examples of such borders acquired by applying a Sobel filter are identified by reference numerals 530 and 540 in FIG. 5. In fitting a model to these edges, information about the directions of the edges, i.e., dark→bright or bright→dark, is useful in distinguishing the appropriate edges from those belonging to other structures. As in contour fitting, the model is adjusted to minimize the RMS distance from the model to the closest edge points. For fitting to edges, however, it is important that the model start close to the solution so as not to be drawn towards inappropriate edges.

In other exemplary embodiments of the current invention different modeling techniques may be used. These techniques include spherical harmonics, Finite Element Methods, and population models.

Once the model with an associated coordinate system is fit, it may serve as an atlas. That is, we now know approximately where the regions of interest lie and we can adjust our scans planes to acquire them accordingly. For example, once a model of the whole heart is fit to a few scout images, the Left Ventricle (“LV”) may be localized in space (the LV is generally of great interest to cardiologists) and further detailed scans may be made of this region. Fewer scan could be dedicated to the less interesting regions such as the Right Atrium (“RA”). In addition, using the whole heart example, once the model is fit, if it is discovered that the RA appears defective (i.e. the image edges does not match well with model/atlas or the atlas had to be deformed in an odd manner) further detailed scans of this region could be called for, to further investigate this inconsistency.

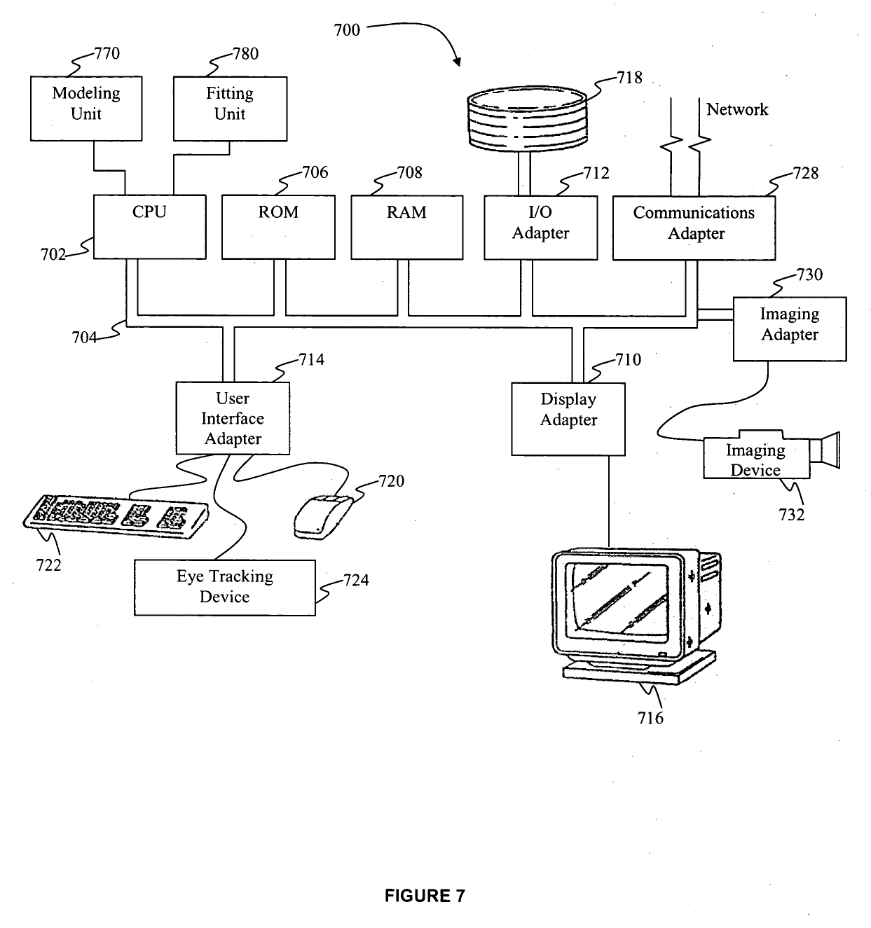

FIG. 7 is schematic diagram of an exemplary embodiment of a system for model assisted planning of medical imaging indicated generally by reference numeral 700. The system 700 includes at least one processor or central processing unit (“CPU”) 702 in signal communication with a system bus 704. A read only memory (“ROM”) 706, a random access memory (“RAM”) 708, a display adapter 710, an I/O adapter 712, a user interface adapter 714, a communications adapter 728, and an imaging adapter 730 are also in signal communication with the system bus 704. A display unit 716 is in signal communication with the system bus 704 via the display adapter 710. A disk storage unit 718, such as, for example, a magnetic or optical disk storage unit, is in signal communication with the system bus 704 via the I/O adapter 712. A mouse 720, a keyboard 722, and an eye tracking device 724 are in signal communication with the system bus 704 via the user interface adapter 714. An imaging device 732 is in signal communication with the system bus 704 via the imaging adapter 730. The imaging device, also know as an acquisition unit, 732 may be a medical imaging device, such as a MR Scanner. The acquisition unit 732 can also be a device for acquiring and reformatting image data, such as the data from CT Volumes.

A modeling unit 770 and a fitting unit 780 are also included in the system 700 and in signal communication with the CPU 702 and the system bus 704. While the modeling unit 770 and the fitting unit 780 are illustrated as coupled to the at least one processor or CPU 702, these components are preferably embodied in computer program code stored in at least one of the memories 706, 708 and 718, wherein the computer program code is executed by the CPU 702. As will be recognized by those of ordinary skill in the pertinent art based on the teachings herein, alternate embodiments are possible, such as, for example, embodying some or all of the computer program code in registers located on the processor chip 702. Given the teachings of the disclosure provided herein, those of ordinary skill in the pertinent art will contemplate various alternate configurations and implementations of the modeling unit 770 and the fitting unit 780, as well as the other elements of the system 700, while practicing within the scope and spirit of the present disclosure.

It is to be understood that the present invention may be implemented in various forms of hardware, software, firmware, special purpose processors, or a combination thereof. In one embodiment, the present invention may be implemented in software as an application program tangibly embodied on a program storage device. The application program may be uploaded to, and executed by, a machine comprising any suitable architecture.

It should also be understood that the above description is only representative of illustrative embodiments. For the convenience of the reader, the above description has focused on a representative sample of possible embodiments, that are illustrative of the principles of the invention, and has not attempted to exhaustively enumerate all possible variations. That alternative embodiments may not have been presented for a specific portion of the invention is not to be considered a disclaimer of those alternate embodiments. Other applications and embodiments can be straightforwardly implemented without departing from the spirit and scope of the present invention. It is therefore intended, that the invention not be limited to the specifically described embodiments, but the invention is to be defined in accordance with that claims that follow. It can be appreciated that many of those undescribed embodiments are within the literal scope of the following claims, and that others are equivalent.

1. A method of medical image acquisition, comprising:
acquiring an image of a region of interest;
acquiring a model of the region of interest; and
fitting the model to the image.

2. A method as defined in claim 1, the method further comprising acquiring at least one new image based on a coordinate system, the coordinate system being associated with the model.

3. A method as defined in claim 2, wherein the new image is part of a standard acquisition.

4. A method as defined in claim 1, wherein the model is created using a technique selected from the group consisting of spherical harmonics, finite element methods, and population models.

5. A method as defined in claim 1, wherein the model type is selected from the group consisting of parametric models and polygonal models.

6. A method as defined in claim 1, the step of fitting the model to the image further comprising:
delineating at least one border of the region of interest in the image; and
fitting the model to the border, with a Root Mean Square distance calculable between the model and the border.

7. A method as defined in claim 6, wherein the delineated border is a contour found in the image.

8. A method as defined in claim 6, wherein the delineated border is an edge found in the image.

9. A method as defined in claim 8, wherein an edge detection algorithm is used to find the edge.

10. A method as defined in claim 9, the edge detection algorithm comprising convolving the image with a filter.

11. A method as defined in claim 6, wherein the model is fit to minimize the Root Mean Square distance.

12. A method as defined in claim 1, the method further comprising acquiring at least one new image based on an inconsistency between the model and the image.

13. A system for medical image acquisition, comprising:
a modeling unit for modeling a region of interest;
an acquisition unit in signal communication with the modeling unit for acquiring a image of the region of interest; and
a fitting unit in signal communication with the acquisition unit for fitting the model to the image.

14. A system as defined in claim 13 wherein the acquisition unit is used to acquire at least one new image based on a coordinate system, and the coordinate system is associated with the model.

15. A system as defined in claim 14, wherein the new image is part of a standard acquisition.

16. A program storage device readable by machine, tangibly embodying a program of instructions executable by the machine to perform a method of medical image acquisition, the program steps comprising:
acquiring an image of the region of interest;
acquiring a model of a region of interest; and
fitting the model to the image.

17. A program storage device as defined in claim 16, the program step of fitting the model to the image further comprising:
delineating at least one border of the region of interest in the image; and
fitting the model to the border, with a Root Mean Square distance calculable between the model and the border.

18. A program storage device as defined in claim 16, the program steps further comprising acquiring at least one new image based on a coordinate system, the coordinate system being associated with the model.

19. A program storage device as defined in claim 18, wherein the new image is part of a standard acquisition.

20. A program storage device as defined in claim 16, the program steps further comprising acquiring at least one new image based on an inconsistency between the model and the image.
